(19) United States
(12) Patent Application Publication
(10) Pub. No.: US 2023/0196576 A1
(43) Pub. Date: Jun. 22, 2023

(54) THERMAL SCANNER FOR JOINT HEALTH

(71) Applicant: GlaxoSmithKline Consumer Healthcare Holdings (US) LLC, Warren, NJ (US)

(72) Inventor: Rishi MULGUND, Warren, NJ (US)

(21) Appl. No.: 18/066,068

(22) Filed: Dec. 14, 2022

Related U.S. Application Data
(60) Provisional application No. 63/291,609, filed on Dec. 20, 2021.

(51) Int. Cl. G06T 7/00; G16H 40/63; G16H 30/40

(52) U.S. Cl. CPC G06T 7/0014; G16H 40/63; G16H 30/40; G06T 2207/10048; G06T 2207/30004; G06Q 30/02

(57) ABSTRACT

A thermal scanning system comprising, a thermal imaging apparatus including a thermal imager configured to generate thermal images of a scanned region of a body of a user, a program including an analysis system to analyze the thermal images to identify areas of inflammation and correlate the inflammation to a joint health of the scanned region, and a screen configured to interface with the user and to communicate the thermal images and joint health information as output.

### CROSS-REFERENCE TO RELATED APPLICATIONS

This application is related to and claims priority from U.S. provisional patent application No. 63/291,609 filed Dec. 20, 2021, the entire contents of which are fully incorporated herein by reference for all purposes.

### FIELD OF THE INVENTION

The present invention relates to a thermal scanning system for evaluating joint health.

### BACKGROUND

Inflammation in the body is a process by which the body protects itself from infection from outside invaders. In healthy individuals, inflammation is limited and ultimately helpful to fight off a foreign substance. Once the infection or virus has been eliminated, inflammation typically resolves. In some diseases, like arthritis, the body triggers inflammation without the threat of outside invaders. In these types of autoimmune diseases, the immune response lacks the ability to self-regulate. As a result, inflammation occurs where it is not needed and can affect the health of joint tissues. Some of the symptoms of inflammation include swollen joints that may be warm to the touch, joint pain, joint stiffness, and a joint that doesn't perform as expected. These symptoms may negatively impact an individual's quality of life.

Known techniques for monitoring inflammation are often inconvenient, expensive, inconclusive, or limited to a hospital setting. Furthermore, these techniques only provide limited information as to joint health. These limitations prevent an individual from monitoring the health of their joints regularly in order to treat and prevent any associated conditions.

Arthritis can often be controlled using medication and, when treated earlier, put in remission. Therefore, there is a need for an accurate, accessible, relatively instant means for measuring inflammation in an individual and providing useful joint health information.

### SUMMARY OF THE INVENTION

Aspects of the present invention are directed to a thermal scanning system comprising: a thermal imaging apparatus including a thermal imager configured to generate thermal images of a scanned region of a body of a user; a program including an analysis system to analyze the thermal images to identify areas of inflammation and correlate the inflammation to a joint health of the scanned region; and a screen configured to interface with the user and to communicate the thermal images and joint health information as output. Aspects of the present invention are further directed to a structure comprising the disclosed thermal scanning system, the structure comprising a beam including a stand at a bottom end and a screen at a top end, wherein the thermal imager is provided on the beam, and a user stands on the stand during operation of the thermal scanning system.

Aspects of the present invention are further directed to a thermal scanning system, wherein the program analyzes the thermal images in substantially real time. Aspects of the present invention are further directed to the program comparing the thermal image with a database of images of various levels of inflammation to determine the relative joint health, including approximate joint age, of the scanned region. The database can be a calibrated database. For example, the images in the database may be graded to levels of inflammation and/or joint health and/or joint age by associating thermal images as produced by the disclosed system with results of another, established clinical method, such as X-ray imaging.

The user begins the process by standing on a stand of the thermal imaging apparatus. Optionally, there may be prompts on a screen requiring the user's response. For example, the user may be asked to input information relating to their age, prior health conditions, the region being scanned, etc.

The thermal imager takes a thermal image of the region through a non-invasive non-contact scan. The image of the region is sent to a computer software or app. Then, the image is analyzed by the software program or application supported by machine learning and artificial intelligence. In an embodiment, the image is compared to a database of preprogramed thermal images to determine the comparative health of the scanned region. In an embodiment, the output from thermal scanning system for each user is an estimated joint age based on the comparative analysis.

In an embodiment, the image topography of the scanned body region can be generated in substantially real time using the positional and/or thermal images acquired by the thermal imager. A complete thermal image of the region of the user may be outputted along with a predicted joint age and/or other details regarding joint health. In an embodiment, the thermal scanning system may output a recommendation for a treatment product, such as Voltaren®, to help with the joint health issues detected.

The output may be displayed on the screen. In another embodiment, the output may be printed or otherwise shared with the user and/or a healthcare professional via email, text message to a mobile device, or sent to an associated application on a mobile device.

### BRIEF DESCRIPTION OF THE DRAWINGS

FIG. 1 is a perspective view of a thermal imaging apparatus according to an embodiment of the invention;

FIG. 2 is a side view of a thermal scanning system in use according to an embodiment of the invention;

FIG. 3 is a schematic block diagram illustrating the components and the interactions between a user and components of the thermal scanning system.

### DETAILED DESCRIPTION

The present invention is directed to a thermal scanning system 10 for conveniently and substantially in real time determining joint age and joint health. The thermal scanning system 10 includes a thermal imaging apparatus 100 and a program as a means of analyzing the thermal images produced by the thermal imaging apparatus 100 using software or an application supported by machine learning and/or artificial intelligence.

Arthritis and other degenerative joint diseases arise from a complex interaction of multiple factors, including local inflammation. The inventors believe that joint surface temperature provides valuable information as to the disease process and health of a joint and may complement radiographic evaluation of arthritis by contributing to augment the static information obtained from x-rays.

In an embodiment, the thermal scanning system 10 may be used outside of the hospital or medical office setting without the need of a medical professional and is a convenient way for users to understand their joint health and their risk for diseases, such as arthritis, that may be caused by inflammation that is measured by the system. This allows users to take proactive steps to protect and/or improve their joint health.

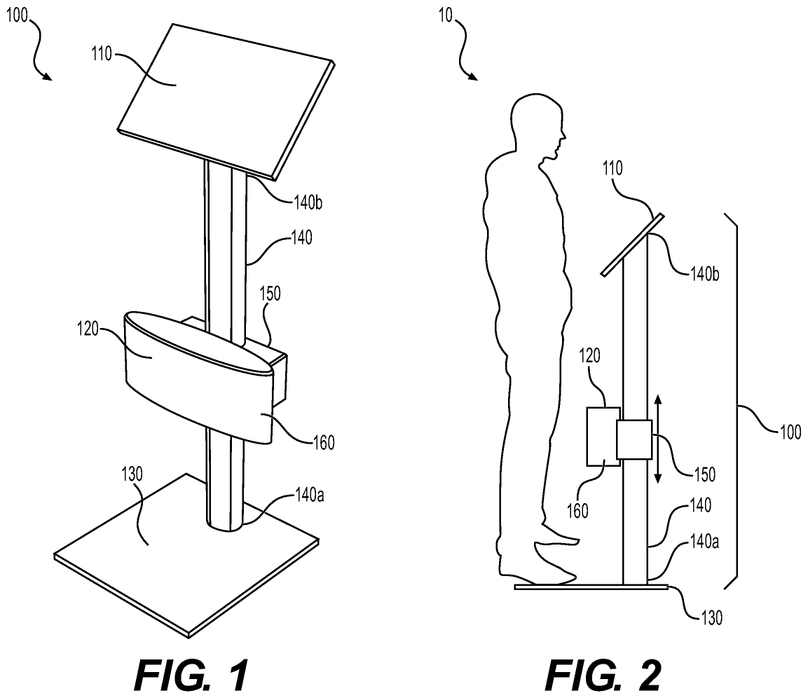

FIG. 1 illustrates a perspective view of a thermal imaging apparatus 100 according to an embodiment. The thermal imaging apparatus 100 is a stationary device that scans regions of the body and generates thermal images to determine levels of inflammation.

The thermal imaging apparatus 100 includes a means for generating thermal images of the region of the body that is scanned. In an embodiment, the thermal imaging apparatus 100 includes a thermal imager 160 to create images of the scanned region using infrared radiation. The thermal imager 160 may be a thermographic camera or an infrared camera. The thermal imager 160 detects and measures the infrared energy of the region scanned and converts the data into an electronic image that shows the approximate surface temperature of the region.

Temperature is a fundamental physical property, and an increase in temperature above normal is one sign of inflammation. The human body has a temperature of about 310° K and radiates primarily in the far infrared spectrum. The thermal imager 160 provides a non-contact, non-invasive means of measuring surface temperature, which helps to calculate the level of inflammation in localized areas. Areas of inflammation may appear as hot spots or areas of inhomogeneity in the thermographic or thermal image generated. The thermographic image is a spatial map of temperatures created by the scanned region of the body.

In an embodiment, the thermographic image is analyzed by the program to determine joint health, joint health risks, and optionally approximate joint age. In an embodiment, the thermographic image generated in a scan is compared with a calibrated database of thermographic images of various levels of inflammation to calculate the relative joint health and/or joint age. This type of comparative analysis provides valuable information regarding an individual's internal inflammation and, ultimately, their joint health.

The thermographic image and the associated health report may be the outputted by the thermal scanning system 10 and may be used by the user to determine appropriate next steps. In an embodiment, the thermal scanning system 10 may recommend the use of over the counter topical cream, gel, or ointment to address the inflammation or the associated symptoms.

In an embodiment, the thermal imager 160 provides a non-contact means of capturing images of the scanned region. In an embodiment, the thermal imager 160 may be between about 2 inches to about 24 inches away from the region to be scanned during operation. In another embodiment, the thermal imager 160 may be between about 6 inches to about 12 inches away from the region to be scanned during operation. Since the thermal imager 160 does not come in contact with the region being scanned, the thermal imaging apparatus 100 can be used by multiple people hygienically without being cleaned or disinfected in between uses.

In an embodiment, the thermal imaging apparatus 100 may be used to scan different areas of the body, for example, the foot, hand, head, knee, or other body parts. In a preferred embodiment, the thermal imaging apparatus 100 is used to scan the knee. In an embodiment, the thermal imaging apparatus 100 includes one or more position sensors to acquire positional data associated of the region scanned.

The thermal imager 160 is secured within a housing 120 to stabilize the thermal imager 160 to eliminate or minimize thermal noise and generate more accurate and consistent images. Further, it may be desirable for the thermal imager 160 to remain a predetermined distance from the region being scanned. The housing 120 can be configured to place the thermal imager 160 at the appropriate predetermined distance. Additionally, the housing 120 protects the thermal imager 160 and keeps it hygienic during multiple uses in a public setting.

The housing 120 is connected to a support beam 140. The support beam 140 extends in a vertical direction and is connected to a stand 130 at a bottom end 140a and a screen 110 at a top end 140b. The support beam 140 may be adjustable in the vertical direction so that the thermal imaging apparatus 100 may be positioned correctly for individuals of varying heights.

The stand 130 may be provided on the floor and a user may stand on the stand during operation of the thermal scanning system 10. The stand 130 may be any suitable shape and size to accommodate the user. In an embodiment, the stand 130 is a flat square. In an embodiment, the stand may include a thermal wind blocker. The thermal wind blocker is a side wall portion that extends upwards from the stand to eliminate thermal nose.

The screen 110 includes the interface and any inputs necessary for operating the thermal scanning system 10. In an embodiment, the screen 110 may be a touch screen. The screen 110 can be used to operate and interact with the thermal scanning system 10 and to provide the state of the device and/or output information such as current temperature reading, the thermal image, joint age/health, suggested treatment plan, among other things. In an embodiment, the screen 110 includes at least a display element, a printed circuit board assembly, one or more sensors, a sensor window, and a source of power such as a battery or power plug. In an embodiment, the display element and the sensors are operatively connected to the printed circuit board assembly.

In an embodiment, a motor 150 is provided on the housing 120 of the thermal imager 160 and is connected to the support beam 140 so that the thermal imager 160 can be easily moved up or down along the length of the support beam 140. In an embodiment, the motor 150 is a manual lever. In another embodiment, the motor 150 is electric. The motor 150 allows the location of the thermal imager 160 to be customized for each user so as to generate accurate images. The motor 150 also allows the thermal imager 160 to be placed appropriately so as to capture different regions of the body.

FIG. 2 illustrates the thermal imaging apparatus 100 in use. A user begins the process by standing on the stand 130 with both feet planted firmly on a surface of the stand 130. Thereafter, the thermal imager 160 is adjusted via the motor 150 in the vertical direction to place the thermal imager 160 directly in front of the region to be scanned. In an embodiment, the thermal imager 160 is placed in front of the knee to scan the knee. The user then turns the system on using the screen 110 and indicates that they would like to begin the thermal scanning process. The program then sends a signal to the thermal imager 160 to begin the scan.

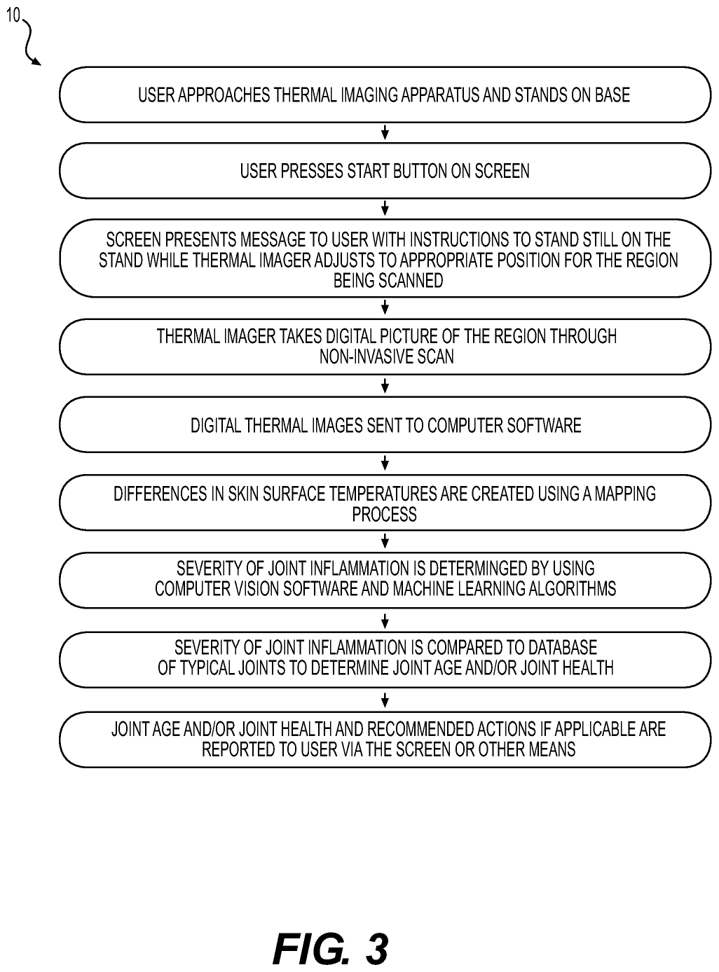

FIG. 3 provides a schematic block diagram illustrating the components and the interactions between a user and components of the thermal scanning system 10. For purposes of the illustrated embodiment, the illustration has been simplified such that many of the components utilized to facilitate operation of the various systems are not shown. One skilled in the relevant art will appreciate that such components can be utilized and that additional interactions would accordingly occur without departing from the spirit and scope of the present disclosure.

The user begins the process by standing on the stand 130 of the thermal imaging apparatus 100 and optionally responding to any prompts on the screen 110. In an embodiment, the user may be asked to input information into the program via the screen 110 such as age, health, concerns, area being scanned, etc.

The thermal imager 160 takes a thermal image of the region through a non-invasive non-contact scan. The image of the region is sent to a computer software or app, the program as described above. Then, the image is analyzed by the software program or application supported by machine learning and/or artificial intelligence. In an embodiment, the image is compared to a database of thermal images to determine the comparative health of the scanned region. In an embodiment, the database of thermal images can be preprogramed. In an embodiment, the output from the thermal scanning system 10 for each user is an estimated joint age based on the comparative analysis.

In an embodiment, the image topography of the scanned body region can be generated in substantially real time using the positional and/or thermal images acquired by the thermal imager 160. A complete thermal image of the region of the user may be outputted along with a predicted joint age and/or other details regarding joint health. In an embodiment, the thermal scanning system may output a recommendation for a treatment product, such as Voltaren®, to help with the joint health issues detected.

The output may be displayed on the screen 110. In another embodiment, the output may be printed or otherwise shared with the user and/or a healthcare professional via email, text message to a mobile device, or sent to an associated application on a mobile device.

In an embodiment, the thermal scanning system 10 is located inside a drugstore, convenience store, or equivalent, where medicine and treatments to improve joint health may be sold. This allows users to use the thermal scanning system 10 to determine their need for such medication and obtain that medication promptly to instantly feel relieve and begin treatment. In an embodiment, the thermal scanning system 10 is located in a setting where users may easily and conveniently access it without any prior approval or scheduling. In an embodiment, the thermal scanning system 10 is mobile and may be easily moved between locations. In an embodiment, no professional medical experience or knowledge is required to use the thermal scanning system 10 or to interpret the output.

What is claimed:

1. A thermal scanning system comprising:
a thermal imaging apparatus including a thermal imager configured to generate thermal images of a scanned region of a body of a user;
a program including an analysis system to analyze the thermal images to identify areas of inflammation and correlate the inflammation to a joint health of the scanned region; and
a screen configured to interface with the user and to communicate the thermal images and joint health information as output.

2. The thermal scanning system according to claim 1, wherein the program analyzes the thermal images in substantially real time.

3. The thermal scanning system according to claim 1, wherein the thermal imager is provided on a beam, the beam including a stand at a bottom end and the screen at a top end, wherein the user stands on the stand during operation of the thermal scanning system.

4. The thermal scanning system according to claim 1, wherein the program compares the thermal image with a database of thermal images of various levels of inflammation to determine a joint health of the scanned region in comparison to the database thermal images.

5. The thermal scanning system according to claim 4, wherein the joint health of the scanned region includes an approximate joint age.

6. The thermal scanning system according to claim 1, wherein the thermal imager is a thermographic camera.

7. The thermal scanning system according to claim 1, wherein the thermal imager is an infrared camera.

8. The thermal scanning system according to claim 3, wherein a motor is provided on a housing for the thermal imager, wherein the motor is connected to the support beam and moves the thermal imager up and down along the length of the support beam.

9. A thermal scanning system for marketing a joint health product according to claim 1.

10. A thermal scanning system for determining joint health according to claim 1.
